(19) United States
(12) Patent Application Publication
(10) Pub. No.: US 2017/0112625 A1
(43) Pub. Date: Apr. 27, 2017

(54) METHOD OF GLENOHUMERAL INSTABILITY REPAIR

(71) Applicant: Smith & Nephew, Inc., Memphis, TN (US)

(72) Inventors: Ettore Taverna, Ligornetto (CH); Jeffrey Wyman, Naples, FL (US); Stephen A Santangelo, Sturbridge, CT (US)

(73) Assignee: Smith & Nephew, Inc., Memphis, TN (US)

(21) Appl. No.: 15/310,651

(22) PCT Filed: Jun. 12, 2015
(86) PCT No.: PCT/US2015/035466

§ 371 Date: Nov. 11, 2016

Related U.S. Application Data
(60) Provisional application No. 62/011,941, filed on Jun. 13, 2014.

(51) Int. Cl. A61F 2/30; A61B 17/17; A61B 17/04; A61F 2/46; A61F 2/40

(52) U.S. Cl. CPC A61F 2/30771; A61F 2/4644; A61F 2/40; A61B 17/0401; A61B 17/1778; A61F 2002/30772; A61B 2017/0404; A61B 2017/0477; A61B 2017/564

(57) ABSTRACT

Methods for arthroscopic bone graft for anterior inferior glenohumeral instability repair are disclosed. The method includes providing a bone graft not attached to soft tissue. Respective passageways are formed through the bone graft and a patient's glenoid. A first implant is further provided, where the first implant is secured to a first end of a suture. The first implant is positioned on the cortical side of the bone graft and the cancellous surface of the bone graft is urged into contact with the anterior surface of the glenoid. A second implant is provided, including an eyelet extending there through. The second end of the suture is inserted through the eyelet of the second implant. The second implant is further placed in contact with the posterior surface of the glenoid. Subsequently, the second end of the suture is secured to the second implant.

### BACKGROUND

The shoulder joint, also referred to as the glenohumeral joint, is the joint between the glenoid cavity (a part of the scapula) and the head of the humerus (upper arm bone). The glenoid cavity is shallow, covering only about a third of the head humeral head. As a result, the glenoid cavity provides relatively little bony constraint upon motion of the humerus and the glenohumeral joint exhibits the widest range of motion of all joints in the human body.

While the glenohumeral joint is also constrained by soft tissue (e.g., cartilage attached to the rim of the glenoid cavity, tendons, etc.), in general, soft tissue cannot provide the same degree of constraint as bone. Accordingly, it is relatively easy to force the humerus from its normal anatomical position with respect to the glenoid socket (i.e., dislocate the shoulder). While not life threatening, a dislocated shoulder can cause pain and immobilization of the joint, impacting a patient's lifestyle.

A variety of procedures have been developed for repair of glenohumeral stability while preserving range of motion. For example, glenohumeral stability may be impaired when a portion of the soft tissue surrounding the rim of the glenoid socket (a mix of fibrous tissue and cartilage referred to as the glenoid labrum) becomes detached from the glenoid socket. In these circumstances, a surgeon may perform a “Bankhart repair,” where the detached portion of the glenoid labrum is reattached to the rim of the glenoid cavity.

However, Bankhart repairs fail to provide sufficient long term stability in circumstances of severe glenoid bone loss. Under these circumstances, stability of the glenohumeral joint is analogous to balancing a golf ball (the humeral head) on a broken tee (the glenoid socket).

In the case of severe bone loss, a surgeon may instead perform a “Latarjet procedure” to repair glenohumeral instability. In a Latarjet procedure, a surgeon attempts to restore bone mass to the glenoid cavity by securing a bone graft to the surface of the glenoid suffering bone loss. When successful, the bone graft acts as a scaffold, allowing the glenoid bone to grow into the bone graft and restore the lost glenoid bone mass (bone fusion). The bone graft is taken from a portion of the patient's scapula referred to as the coracoid process or simply coracoid, with muscles still attached to the coracoid. Thus, when the coracoid graft is fused to the glenoid cavity, the muscles attached to the coracoid provide further constraint upon the glenohumeral joint.

Viewed in a continuum of care, Bankhart repairs are considered a relatively non-aggressive form of treatment, as they “anatomic,” preserving the patient's anatomy in its natural position. In contrast, Latarjet procedures are considered a relatively aggressive form of treatment, as the transfer of the coracoid to the glenoid is non-anatomic. For example, in addition to repositioning a portion of the coracoid and attached muscles, Latarjet procedures also require forming a channel through the subscapularis muscle interposed between the coracoid and glenoid cavity in order to achieve positioning of the coracoid upon the glenoid. Latarjet procedures have historically provided a high success rate for repairing glenohumeral stability due to glenoid bone loss and, therefore, have become a popular course of treatment under these circumstances.

Notably, however, treating glenohumeral instability due to glenoid bone loss in the first instance with a Latarjet procedure goes counter to the general preference of surgeons to follow an escalating course of treatment, beginning with less aggressive procedures before moving to more aggressive procedures.

### SUMMARY

Accordingly, there is a need for less aggressive (e.g., anatomical) surgical procedures for repairing glenohumeral stability and that also provide long-term stability of the glenohumeral joint. Embodiments of the present disclosure are directed to methods for anterior inferior glenohumeral instability repair.

As discussed in greater detail below, the method may include providing a bone graft having a cortical side and a cancellous side, wherein the bone graft is not attached to soft tissue; forming at least one passageway through the bone graft; forming at least one passageway through a glenoid, from a posterior surface to an anterior surface; providing a first implant secured to a first end of a suture; positioning the first implant on the cortical side of the bone graft; drawing the suture posteriorly, through the bone graft passageway and the glenoid passageway, wherein after the suture is so drawn, a second end of the suture is positioned on the posterior side of the glenoid, the first implant contacts the cortical side of the bone graft, and the cancellous surface of the bone graft is urged into contact with the anterior surface of the glenoid; providing a second implant including an eyelet extending there-through; inserting the second end of the suture through the eyelet of the second implant;
positioning the second implant in contact with the posterior glenoid surface; and securing the second end of the suture to the second implant.

Further embodiments of the method may include one or more of the following, alone or in combination. The method may further comprise forming a posterior portal to allow access to the glenoid. The bone graft may be an allograft. The bone graft may not be a coracoid process. The bone graft may have a length between about 10 mm to about 20 mm, a width between about 8 mm to about 10 mm and a thickness of about 5 mm to about 11 mm. Forming least one passageway through the bone graft may comprise drilling at least one passageway through the bone graft with a drill. The at least one passageway through the bone graft may be two passageways through the bone graft. The two passageways through the bone graft may be spaced about 10 mm apart. The at least one passageway through the bone graft may have a diameter of about 3 mm. Forming at least one passageway through the glenoid may comprise drilling at least one passageway through the glenoid with a drill. The drill may be a 2.8 mm sleeved drill. The at least one passageway through the glenoid may be two passageways through the glenoid. The two passageways through the glenoid may be spaced about 10 mm apart. A distance between the at least one passageway through the bone graft and another passageway through the bone graft may be substantially equal to a distance between the at least one passageway through the glenoid and another passageway through the glenoid. The at least one passageway through the glenoid may be placed about 4 mm to about 5 mm on center below a cortical edge of the anterior surface of the glenoid. Drilling at least one passageway through the glenoid with a drill may comprise passing the drill through a billet secured to the posterior surface of the glenoid. Securing the billet to the posterior surface of the glenoid may comprise securing the billet with a glenoid guide. Drawing the suture posteriorly may comprise drawing a guide wire to which the suture has been secured posteriorly. Securing the second end of the suture to the second implant may comprise tying a knot in the suture. The knot may be a Nice Knot. The second implant may be a round endbutton.

Beneficially, in contrast to Latarjet procedures, embodiments of the bone graft may be a bone taken from the patient (autograft) or a donor (allograft). Furthermore, embodiments of the bone graft are not required to be taken from the patients shoulder. Additionally embodiments of the bone graft are not required to be attached to any tissue. For example, embodiments of the bone graft may include, but are not limited to, a portion (e.g., tip) of the iliac crest, the spine of the scapula, etc. As a result, glenohumeral instability due to glenoid bone loss may be repaired in a less aggressive, anatomic manner, preserving the option to perform a more aggressive Latarjet procedure subsequently, if necessary.

### BRIEF DESCRIPTION OF THE DRAWINGS

The foregoing and other objects, features and advantages will be apparent from the following more particular description of the embodiments, as illustrated in the accompanying drawings in which like reference characters refer to the same parts throughout the different views. The drawings are not necessarily to scale, emphasis instead being placed upon illustrating the principles of the embodiments.

FIG. 1 is a photograph illustrating a patient constrained for glenohumeral repair surgery;

FIGS. 2-3 are images of a glenohumeral joint under preparation for repair according to embodiments of the disclosed repair operation;

FIG. 4 is an image of a glenohumeral joint illustrating insertion of a spinal needle during repair according to embodiments of the disclosed repair operation;

FIGS. 5-11 are schematic illustrations of a patient's glenoid and glenoid guide illustrating positioning of a glenoid drill guide during repair according to embodiments of the disclosed repair operation;

FIGS. 12-13B are schematic illustrations of preparation of a bone graft for use in embodiments of the disclosed repair operation;

FIGS. 14-22 are schematic illustrations showing passage of the graft and loading of an implant to secure the graft in position in embodiments of the disclosed repair operation;

FIGS. 23A-23F are images illustrating formation of a Nice Knot in embodiments of the disclosed repair operation;

FIGS. 24-26 are schematic illustrations showing a Nice Knot being secured in embodiments of the disclosed repair operation;

FIG. 27 is a schematic illustration showing the bone graft secured to the glenoid upon completion of embodiments of the disclosed repair operation.

### DETAILED DESCRIPTION

Embodiments of the present disclosure will now be discussed with reference to the figures.

In the description that follows, like components have been given the same reference numerals, regardless of whether they are shown in different examples. To illustrate example(s) in a clear and concise manner, the drawings may not necessarily be to scale and certain features may be shown in somewhat schematic form. Features that are described and/or illustrated with respect to one example may be used in the same way or in a similar way in one or more other examples and/or in combination with or instead of the features of the other examples.

Comprise, include, and/or plural forms of each are open ended and include the listed parts and can include additional parts that are not listed. And/or is open ended and includes one or more of the listed parts and combinations of the listed parts.

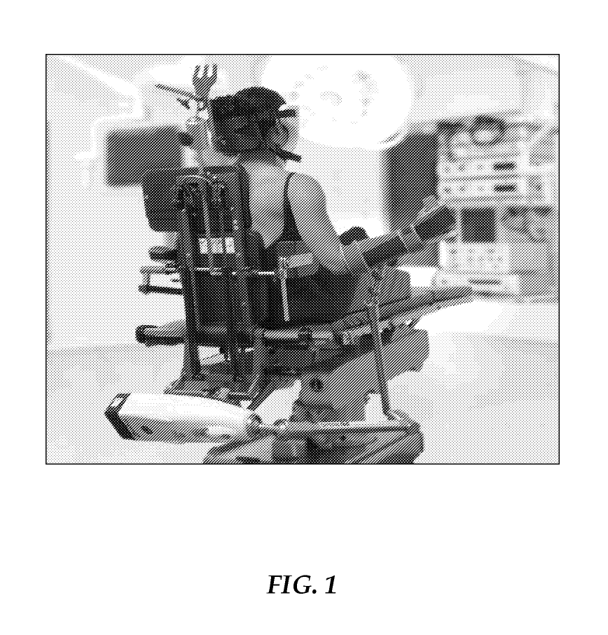

The discussion will now turn to the figures, beginning with FIG. 1-3, illustrating patient positioning and preparation of the glenohumeral joint for repair according to embodiments of the disclosure. FIG. 1 illustrates a photograph of a patient positioned prior to surgery. In certain embodiments, the patient may be positioned in the beach chair or lateral decubitus position. The scapula may be bolstered to externally rotate the glenoid. Draping may allow ready access to the posterior and anterior aspects of the shoulder girdle.

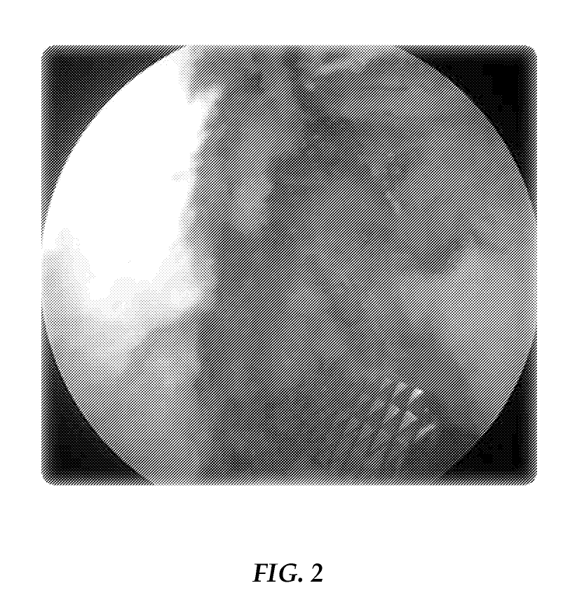

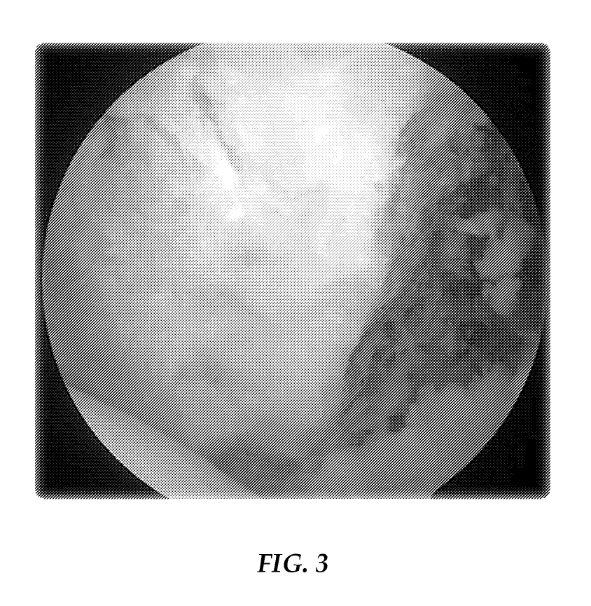

FIGS. 2-3 are images of the embodiments of the glenohumeral joint. In the embodiment of FIG. 2, the labrum and capsule are elevated through abrasion and debridement of the anterior glenoid neck. In the embodiment of FIG. 3, a small area of articular cartilage along the anterior glenoid edge is debrided to allow recognition of the glenoid cortex.

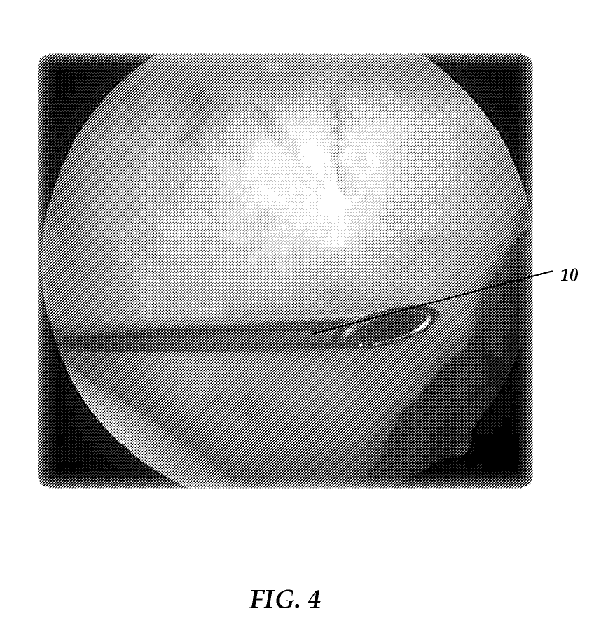

The discussion will now turn to FIGS. 4-11, illustrating embodiments of glenoid guide and drill pin placement. A posterior portal is formed in the body to allow access to the glenoid face. For example, a needle 10 (FIG. 4) is inserted from posterior to anterior, along the face of the glenoid and below the midline, to form the portal. In further embodiments, a medial portal is made to provide access to the glenoid face.

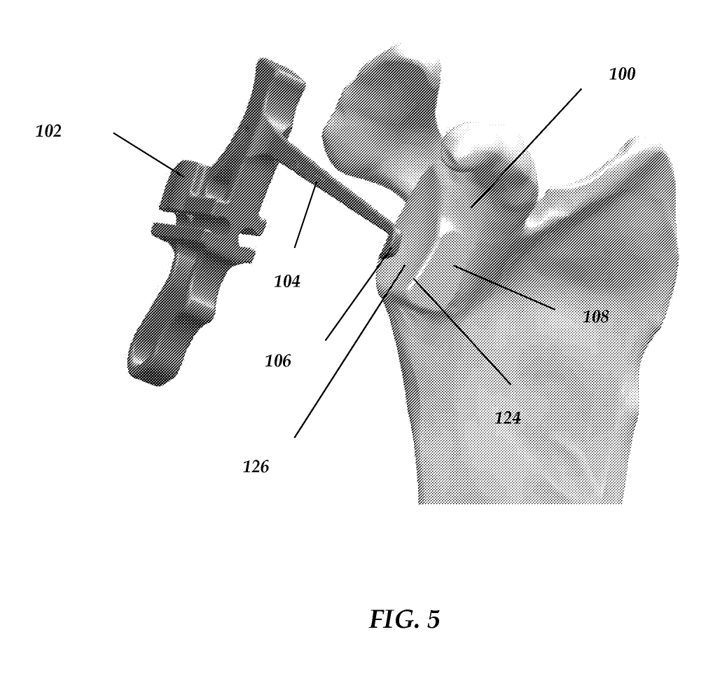

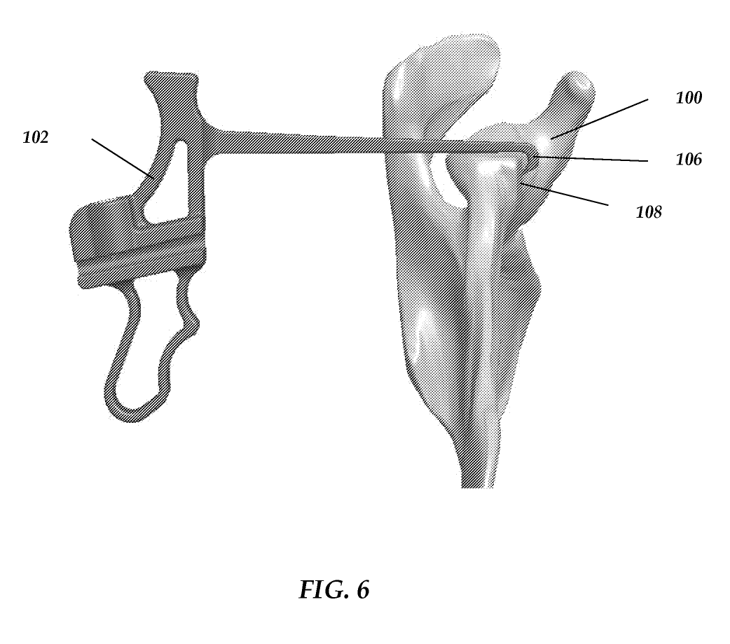

FIGS. 5-11 are schematic illustrations of a patient's glenoid 100 illustrating positioning of a glenoid guide 102 during repair according to embodiments of the disclosed repair operation. In the embodiment of FIG. 5, the glenoid guide 102 includes an arm 104 having a hook 106 formed in the distal end. The hook 106 of the glenoid guide 102 is inserted through the posterior portal (not shown). The hook 106 is passed along the glenoid 100, with the arm 104 of the glenoid guide 102 parallel to the glenoid face 126 in order to avoid damage of the articular surface. The hook 106 is further passed over the cortical edge 124 of the glenoid 100. With reference to the embodiment of FIG. 6, once the glenoid guide 102 is sufficiently advanced, the glenoid guide 102 is rotated to capture the anterior surface 108 of the glenoid 100. In certain embodiments, the hook 106 may be placed at the position that correlates with the mid-point of a bone graft, as further described below.

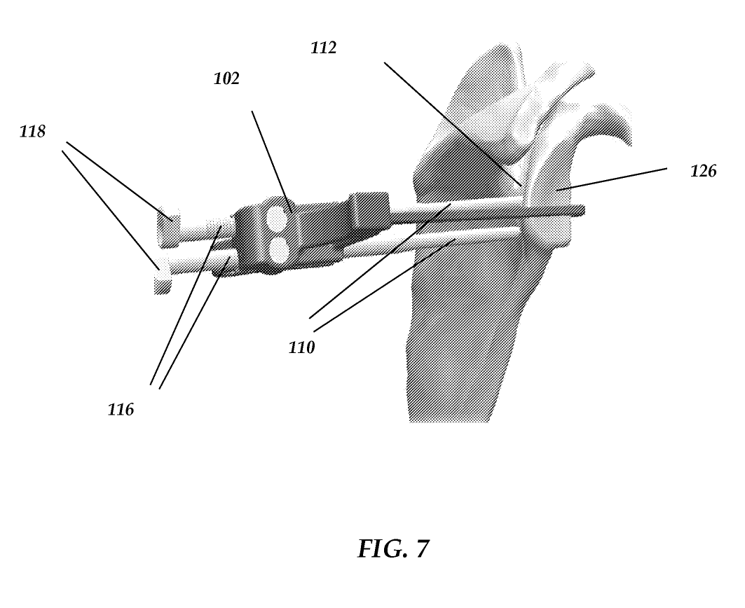

With regards to the embodiment of FIG. 7, the glenoid guide 102 is secured in place. For example, in an embodiment, the glenoid guide 102 is secured in place by two bullets 110 placed percutaneously. A small skin incision is made and a bullet 110 may be advanced until it firmly contacts the posterior surface 112 of the glenoid 100. In certain embodiments, ratcheted teeth 116 of the bullets 110 are aligned with the screws 118 adjacent to a handle of the glenoid guide 102. This process may be repeated for the second bullet 110. In further embodiments, each bullet 110 may be approximately parallel to one another. In additional embodiments, the bullets 110 may be separated by a selected distance (e.g., 10 mm).

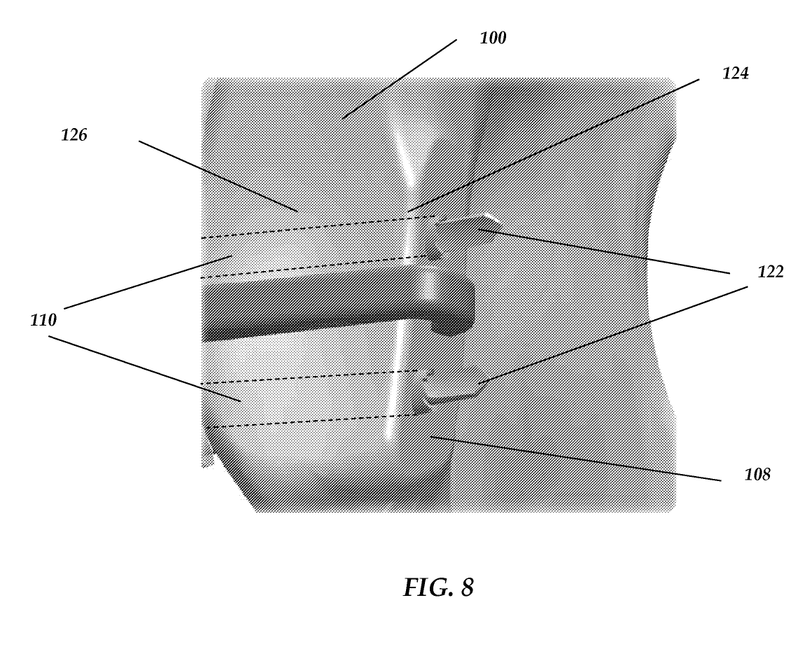

With regards to the embodiment of FIG. 8, a drill is placed in the glenoid guide 102 (not shown). For example, in an embodiment, a sleeved drill (e.g., a 2.8 mm sleeved drill) 122 is placed in each bullet 110 and advanced until exiting from the anterior surface 108 of the glenoid 100. Each drill 122 may be placed a selected distance (e.g., about 4 to about 5 mm) on center below the cortical edge 124 of the anterior surface 108.

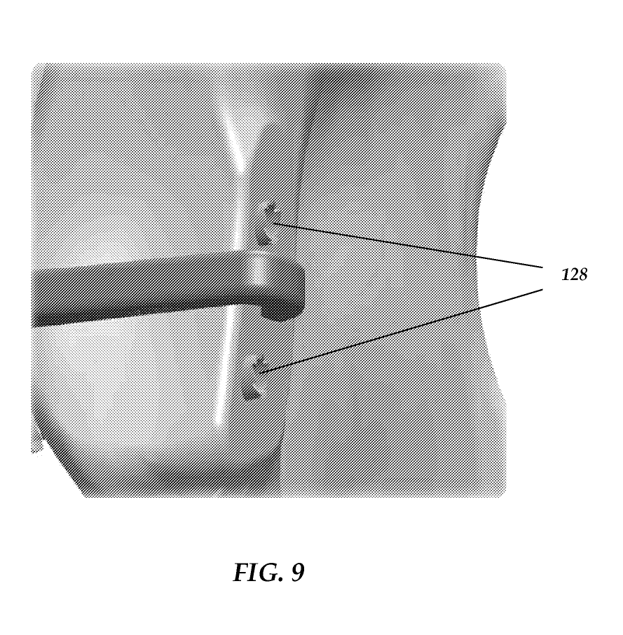

With regards to the embodiment of FIG. 9, the drill is removed. For example, the sleeved drill 122 is removed, leaving the cannulated outer sleeve 128. The presence of arthroscopic fluid exiting posteriorly from the outer sleeve 128 may be employed as confirmation of safe positioning.

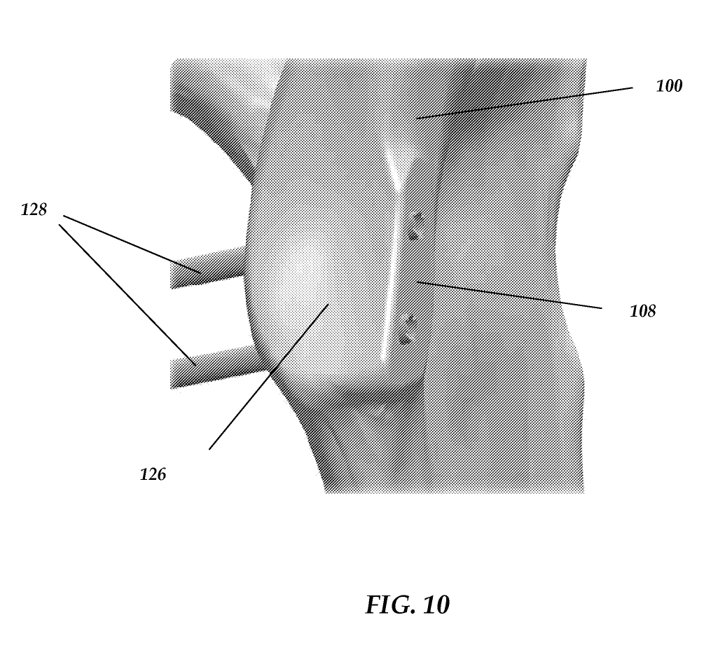

With regards to the embodiment of FIG. 10, the bullets 110 and glenoid guide 102 (not shown) are removed once drilling is complete. For example, in an embodiment, the bullets 110 may be removed by rotating each bullet 110 to disengage the ratcheted teeth 116 of the bullets 110 and extracting the bullets 110 posteriorly. In a further embodiment, the hook 106 (not shown)of the glenoid guide 102 may be disengaged from the glenoid 100. For example, the glenoid guide 102 may be rotated such that the hook 106 is flat against the glenoid surface 126. Should the hook 106 become caught on the posterior labrum, the handle of the glenoid guide 102 may be dropped (or raised, depending on the orientation of the hook) while retrieving. During removal of the bullets 110 and glenoid guide 102, the outer sleeves 128 are left in place, firmly positioned within the glenoid 100.

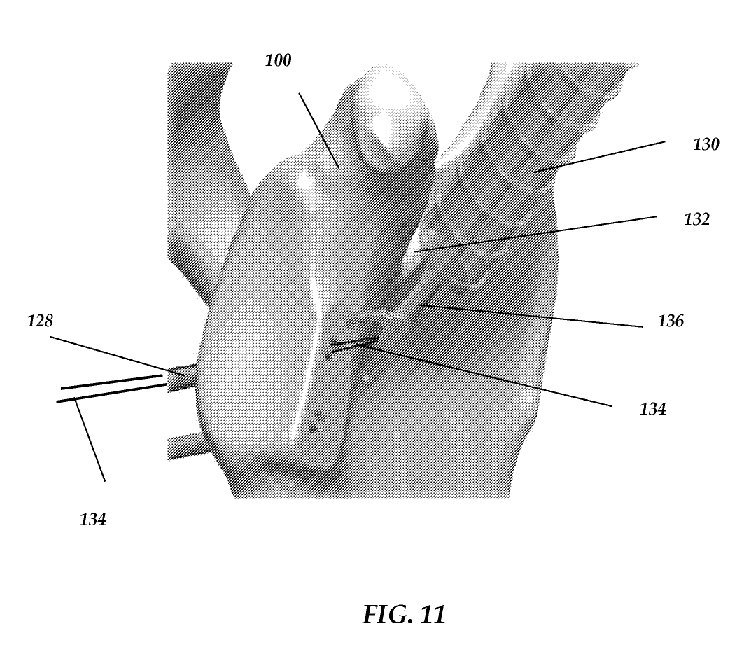

With regards to the embodiment of FIG. 11, a cannula 130 is introduced through the rotator interval region of the patient's shoulder 132. For example, in an embodiment, a 10 mm cannula 130 is introduced through the rotator interval 132. Flexible looped guide wires 134 enter the outer sleeves 128 by passing one guide wire 134 through each outer sleeve 128, posterior to anterior. Each guide wire 134 is subsequently retrieved using a loop grasper 136 passed through the cannula 130. The guide wires 134 are separated and stored (i.e., set apart). After completing storage of the guide wires 134, the outer sleeves 128 may be removed from the glenoid 100.

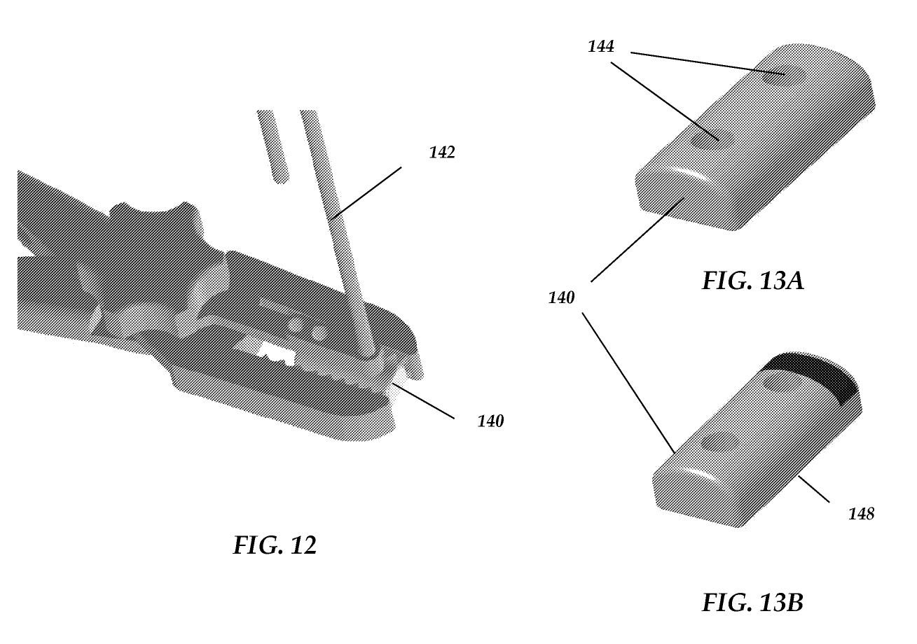

FIGS. 12-13B are schematic illustrations of preparation of a bone graft 140 for use in embodiments of the disclosed repair operation. In an embodiment, the dimensions of the bone graft 140 and the type of bone are selected. For example, in an embodiment, the dimensions of the bone graft 140 may be about 20 mm long by about 8 mm wide by about 8 mm thick. In a further embodiment, a bone graft 140 having the selected dimensions may be prepared from the patient's tricortical iliac crest (autograft) or donor (allograft) material. However, it may be understood that, in alternative embodiments, the bone graft 140 may be taken from other bones. In further embodiments, the prepared bone graft 140 is not attached to soft tissue (e.g., tendon, etc.), in contrast to Latarjet procedures or other bone-tissue-bone type grafts.

In further embodiments, the size of the bone graft 140 may be varied. For example, the bone graft 140 may be approximately 10 mm long by 10 mm wide or larger. The thickness of the bone graft may be about 5 mm to about 11 mm. In another embodiment, the cross-sectional area of the bone graft 140 may be dimensioned smaller than the cross-sectional area of a cannula through which the bone graft 140 is passed. In additional embodiments, the bone graft 140 may be passed into the patient's anatomy using a half cannula so as to not limit the size of the bone graft 140 to a constraining dimension. The bone graft 140 may be fashioned utilizing a graft master preparation board (not shown).

With further regards to the embodiments of FIGS. 12-13B, a drill guide 142 is employed to drill a plurality of holes or passageways 144 at selected locations within the bone graft 140. For example, in an embodiment, two holes 144 having a diameter of about 3 mm may be formed using a drill guide (e.g., Kirschner wire or “K-wire”) that are about 10 mm apart and about 5 mm from each edge of the bone graft 140. The drill guide 142 enters through the cortical side 146 and exits the cancellous side 148 of the bone graft 142. The created holes 144 may correspond to the outer drill sleeves 128 previously placed on the glenoid 100 (FIG. 11). With a marking member (e.g., an ink marker), the cortical side 146 of the bone graft 140 is marked (FIG. 13B). The cancellous side 148 of the bone graft 140 will be secured against the anterior surface 108 of the glenoid 100.

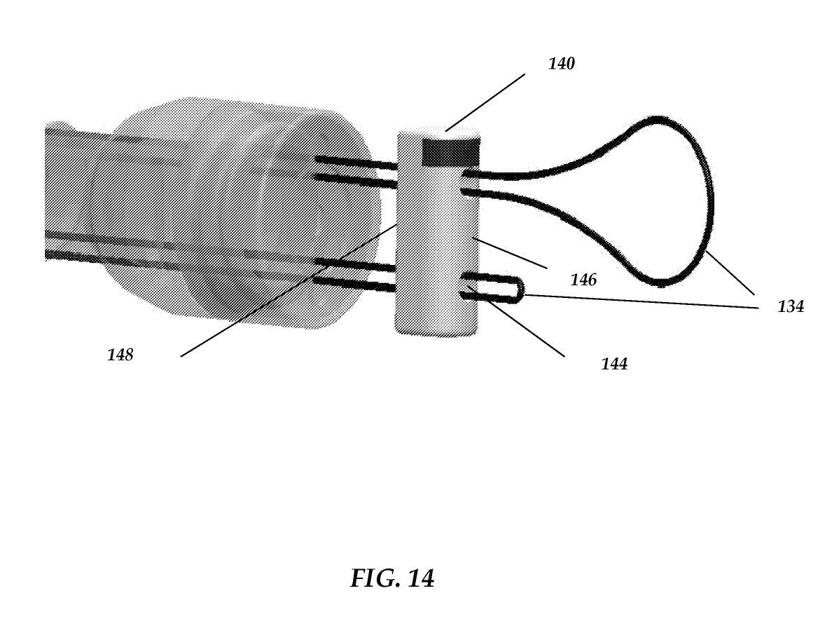

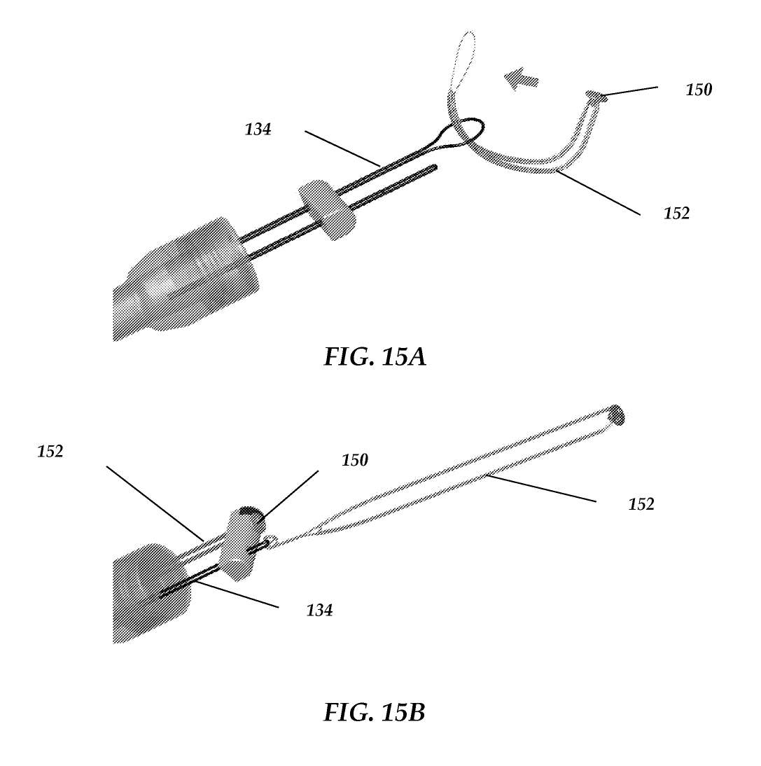

With further regards to the embodiments of FIGS. 14-22, passage of the bone graft 140 and loading of an anterior implant 150, which may be a button, to secure the bone graft 140 in position in embodiments of the disclosed repair operation is illustrated. In the embodiment of FIG. 14, prior to loading the anterior implant 150 (FIG. 15) onto the guide wires 134, care is taken to insure that the looped guide wires 134 are not tangled within the joint. Each looped guide wire 134 is inserted into the passageways 144 of the prepared bone graft 140 on the cancellous side 148 and exits on the cortical side 146. As shown in FIG. 14, the guide wires 134 may have different shapes depending on how much they are pushed into the joint. In the embodiments of FIGS. 15A-15B, the anterior implant 150 is secured to the end of the loop guide wire 134 (e.g., with a classic slip knot). This operation may be achieved by passing the loop of the lead suture 152 through the looped guide wire 134 and feeding the anterior implant 150 through the lead suture loop 152.

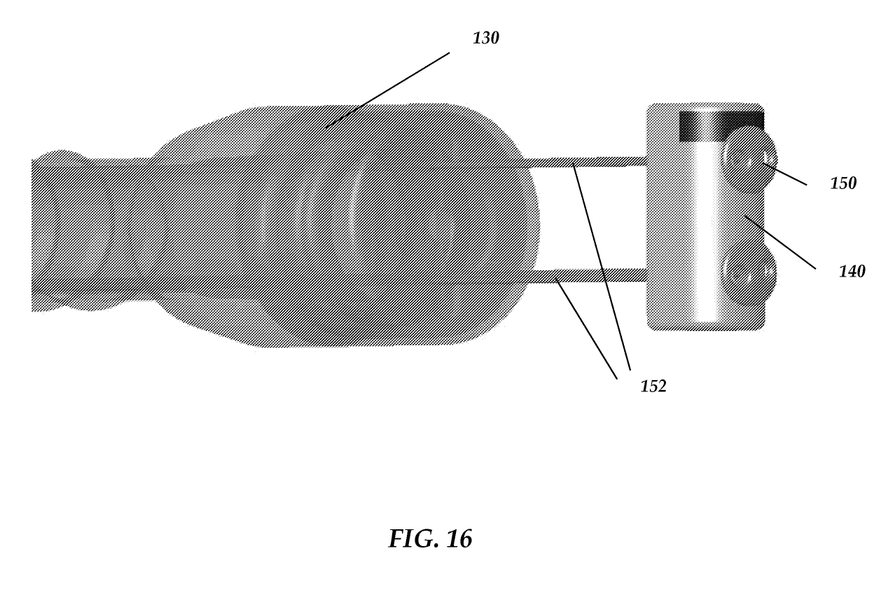

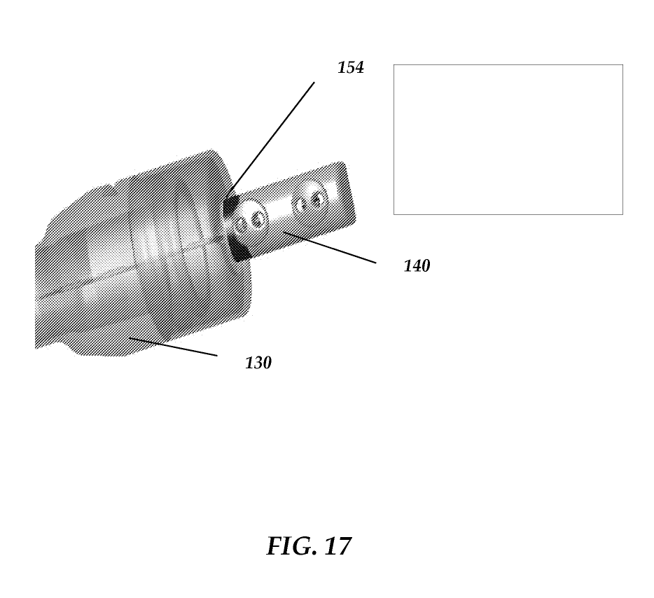

In the embodiment of FIGS. 16-17, the guide wires 134 (not shown) are retracted and the bone graft 140 is inserted within the cannula 130. For example, in an embodiment (FIG. 16), the bone graft 140 is inserted into the cannula 130 within the rotator interval and the guide wires 134 are withdrawn posteriorly to engage the anterior implant 150 and remove slack. This operation allows for smooth movement of the bone graft 140 down the cannula 130. In a further embodiment (FIG. 17), the bone graft 140 is tipped for insertion into the cannula 130 (e.g., a 10 mm cannula). Care may be taken to ensure that the superior end 154 of the bone graft 140 enters the cannula 130 first.

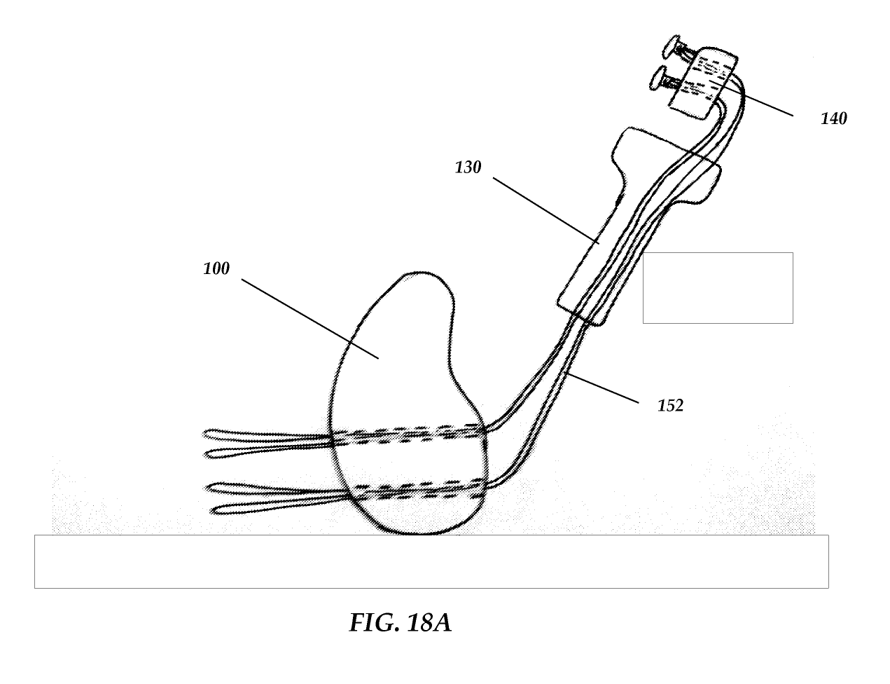

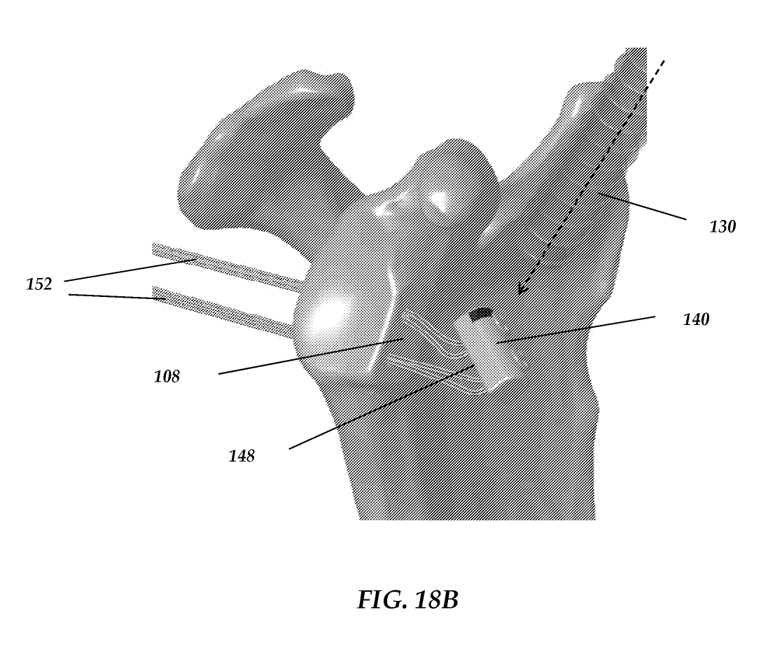

In the embodiment of FIGS. 18A-B, the bone graft 140 is advanced through the cannula 130. For example, in the embodiment of FIG. 18A, the bone graft 140 is advanced through the cannula 130 by pushing it down using a prosthetic device, such as an obterator (not shown). In the embodiment of FIG. 18B, the bone graft 140 is set into position by pulling the guide wires 134 (not shown) posteriorly. The bone graft 140 may be oriented so that the cancellous side 148 is approximately perpendicular to the anterior surface 108 of the glenoid 100. In further embodiments, slight tension may be maintained on the guide wires 134 during advancement of the bone graft 140 through the cannula 130.

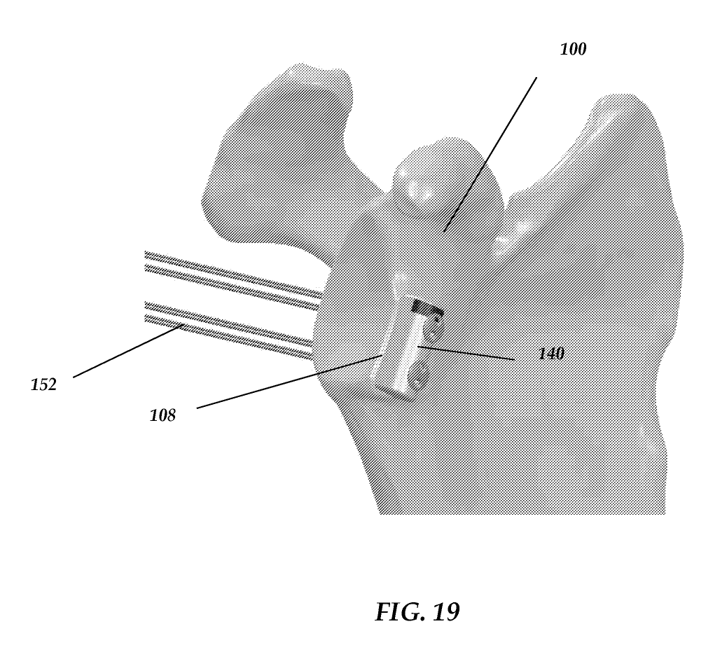

In the embodiment of FIG. 19, the bone graft 140 is positioned on the glenoid 100. For example, in an embodiment, the guide wires 134 (no shown) are drawn posteriorly until the bone graft 140 sits approximately flush on the anterior surface 108 of the glenoid 100. A suture (e.g., a size 00 suture) attached to the suture loop 152 is pulled posteriorly, through the skin. In certain embodiments, the suture 152 may be a continuous loop of suture. Subsequently, the continuous loop of suture 152 is cut to separate the two ends of the continuous loop.

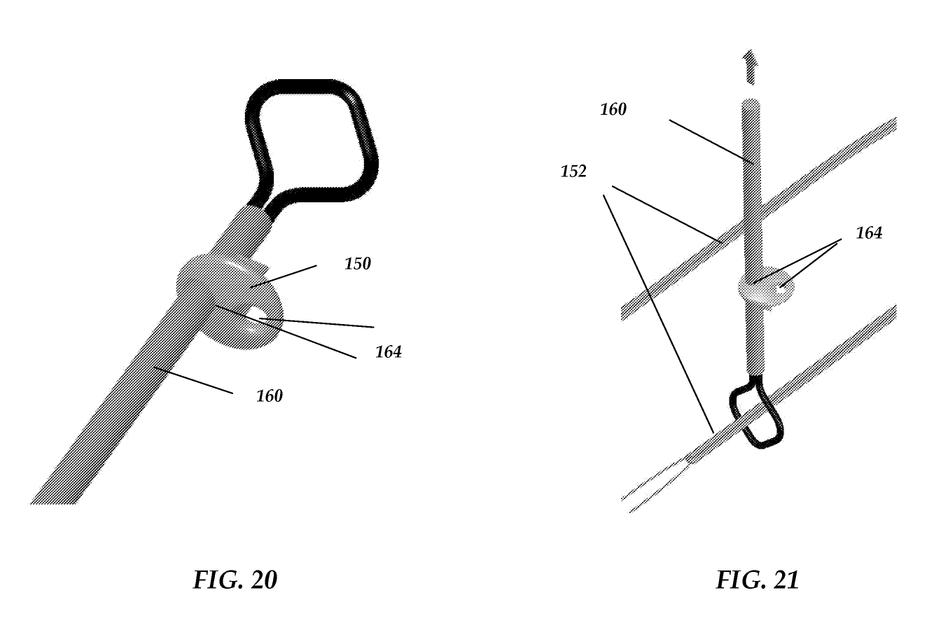

In the embodiment of FIGS. 20-21, the posterior implant 150 is loaded. In an embodiment, the posterior implant 150 is a round endobutton having a plurality of eyelets 162. Non-limiting examples of the round endobutton may be found in U.S. patent application Ser. No. 13/213,966, the entirety of which is hereby incorporated by reference. For example, in an embodiment (FIG. 20), the posterior implant 150 is loaded by advancing an instrument 160 (e.g. a transporter) through an eyelet 164 of the posterior implant 150. In a further embodiment (FIG. 21), the suture 152 is passed through the instrument 160. The instrument 160 is retracted to allow the suture 152 to pass through the eyelet 164 of the posterior implant 150. The operations of FIGS. 20-21 are repeated for the second eyelet 164 with the other side of the suture 152.

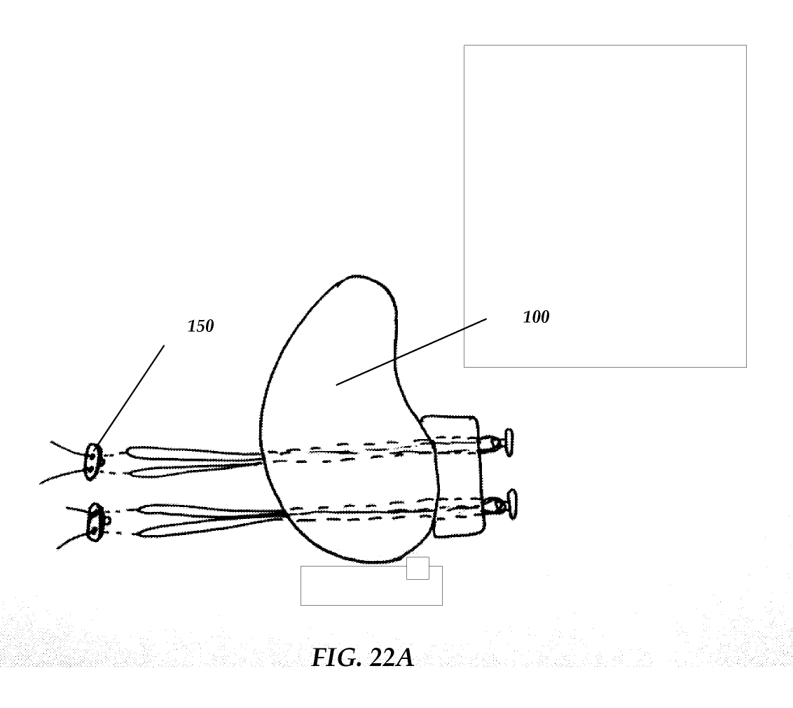

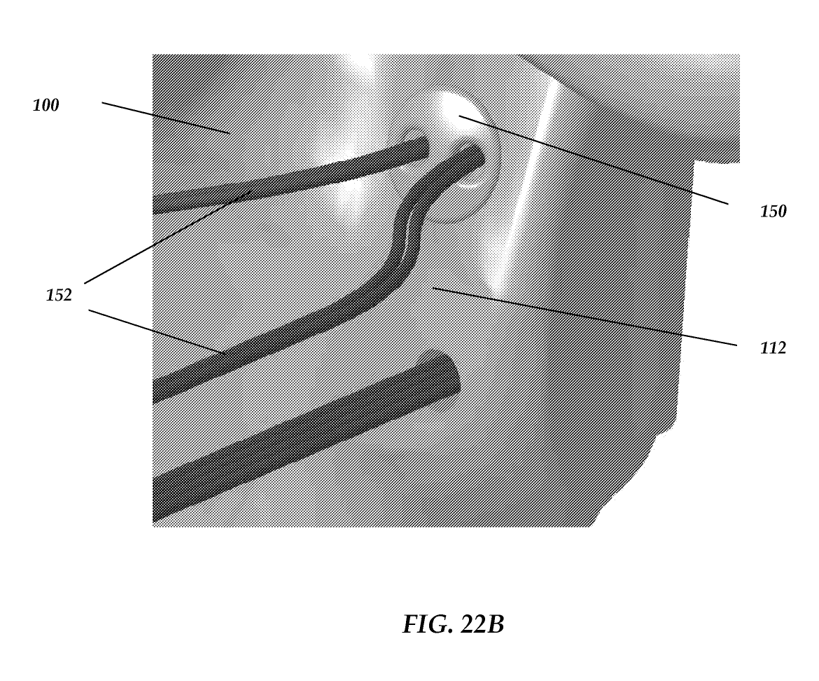

In the embodiment of FIGS. 22A-B, the posterior implant 150 is advanced into contact with the glenoid 100. For example, in an embodiment, the posterior implant 150 is advanced to sit approximately flush against the posterior surface 112 of the glenoid 100 using a knot pusher (not shown). Beneficially, the knot pusher may provide tactile feedback when the posterior implant 150 is properly seated.

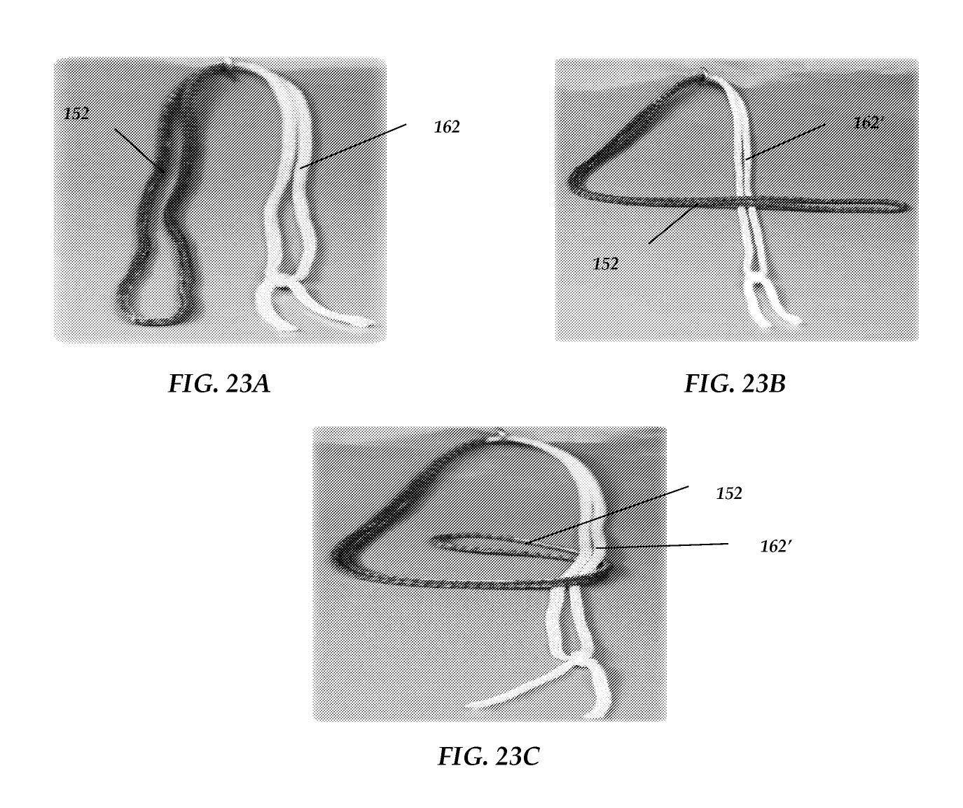

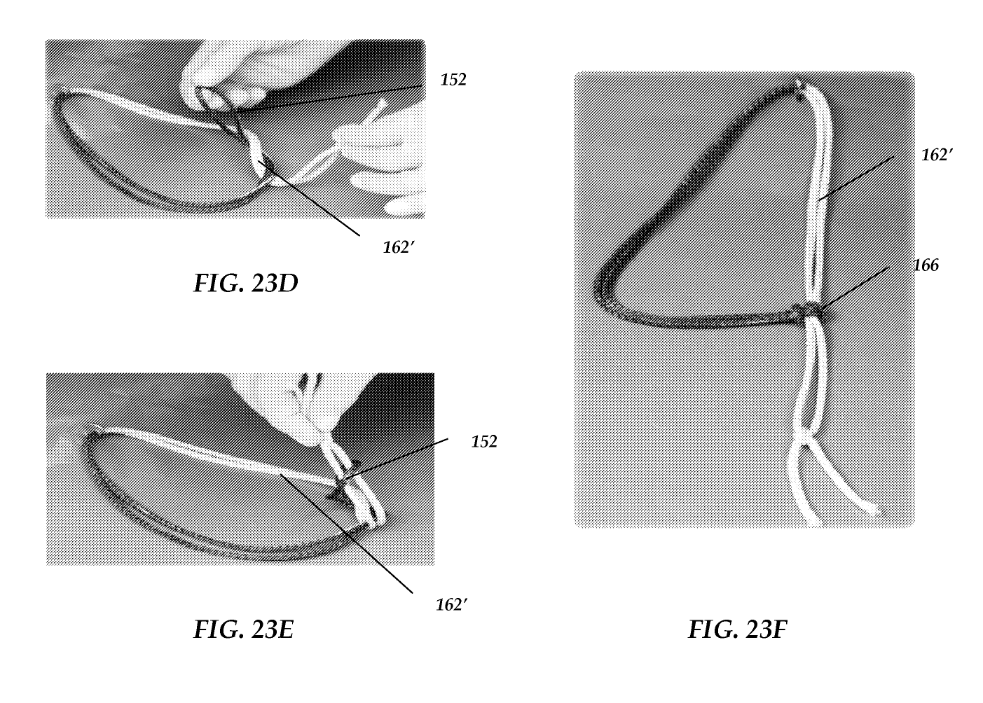

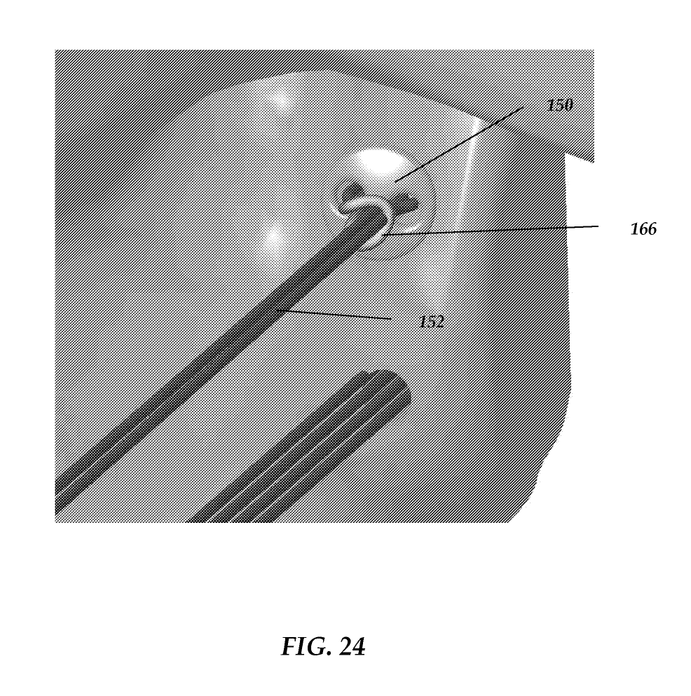

FIGS. 23A-23F are images illustrating formation of a “Nice Knot” 166 (FIG. 23F) in embodiments of the disclosed repair operation. In the embodiment of FIG. 23A, the side of the continuous suture loop 152 with the attached size 00 suture 162 will function as a post 162′. In the embodiment of FIG. 23B, with the post 162′ in the surgeon's dominant hand, a figure four is created by placing the suture loop 152 over the post 162′. In the embodiments of FIG. 23C-23E, the suture loop 152 is brought underneath the post 162′ and through the figure four. The suture loop 152 is subsequently opened at its end. The post 162′ is then placed through the suture loop 152. In the embodiment of FIG. 23F, care is taken to ensure that the Nice Knot 166 is fully taut prior to pulling the post 162′ and advancing the posterior implant 150.

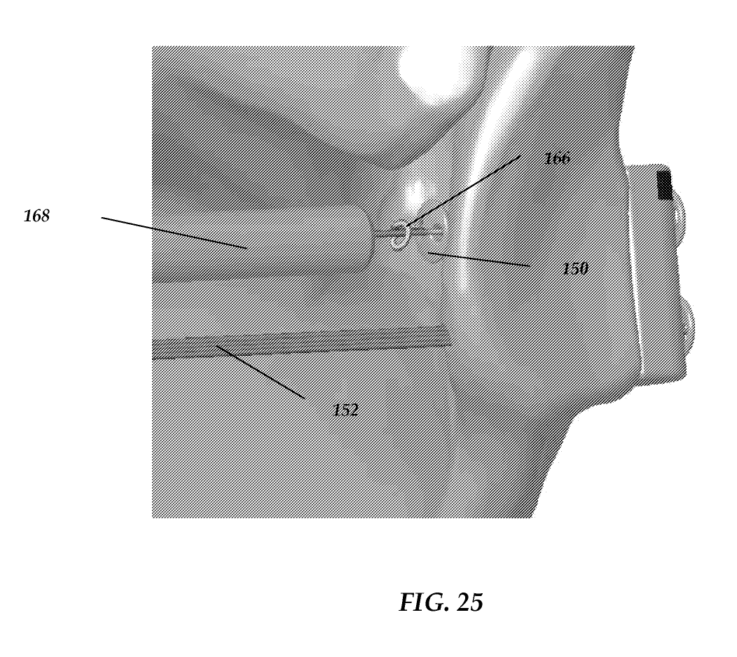

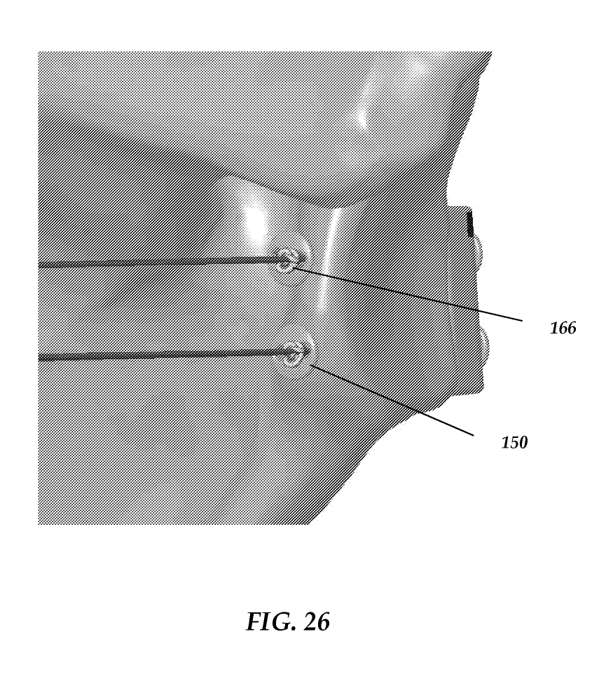

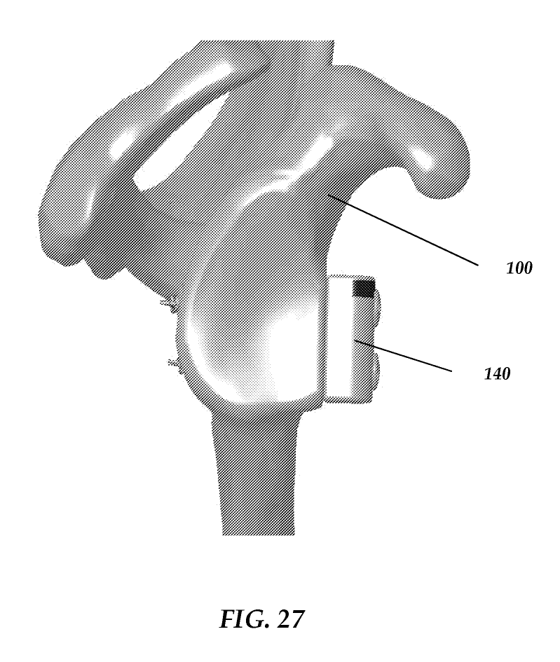

FIGS. 24-26 are schematic illustrations showing a Nice Knot 166 being secured in embodiments of the disclosed repair operation. In the embodiment of FIG. 24, the Nice Knot 166 is advanced to the face of the posterior implant 150 by pulling on the post 162′ (not shown). In the embodiment of FIG. 25, a tensioner 168 may be employed to better secure the posterior implant 150. After the posterior implant 150 has been tensioned, it may be secured with further knots (e.g., half-hitch knots). Once the posterior implant 150 has been secured, the remaining suture 152 may be cut. In the embodiment of FIG. 26, the operations illustrated in FIGS. 23A-25 may be repeated for other posterior implants 150. In another embodiment, the Nice Knot 166 may be a sliding, locking knot.

FIG. 27 is a schematic illustration showing the bone graft 140 secured to the glenoid 100 upon completion of embodiments of the disclosed repair operation.

One skilled in the art will realize the invention may be embodied in other specific forms without departing from the spirit or essential characteristics thereof. The foregoing embodiments are therefore to be considered in all respects illustrative rather than limiting of the invention described herein. Scope of the invention is thus indicated by the appended claims, rather than by the foregoing description, and all changes that come within the meaning and range of equivalency of the claims are therefore intended to be embraced therein.

1. A method of glenohumeral instability repair, comprising:
forming at least one passageway through a bone graft, the bone graft having a cortical side and a cancellous side, wherein the bone graft is not attached to soft tissue;
forming at least one passageway through a glenoid, from a posterior surface to an anterior surface;
positioning a first implant secured to a first end of a suture on the cortical side of the bone graft;
drawing the suture posteriorly, through the bone graft passageway and the glenoid passageway, wherein after the suture is so drawn, a second end of the suture is positioned on the posterior side of the glenoid, the first implant contacts the cortical side of the bone graft, and the cancellous surface of the bone graft is urged into contact with the anterior surface of the glenoid;
inserting the second end of the suture through an eyelet of a second implant;
positioning the second implant in contact with the posterior glenoid surface; and
securing the second end of the suture to the second implant.

2. The method of claim 1, further comprising forming a posterior portal to allow access to the glenoid.

3. The method of claim 1, wherein the bone graft is an allograft.

4. The method of claim 1, wherein the bone graft is not a coracoid process.

5. The method of claim 1, wherein the bone graft has a length between about 10 mm to about 20 mm.

6. The method of claim 1, wherein the bone graft has a width between about 8 mm to about 10 mm.

7. The method of claim 1, wherein the bone graft has a thickness of about 5 mm to about 11 mm.

8. The method of claim 1, wherein forming at least one passageway through the bone graft comprises drilling at least one passageway through the bone graft with a drill.

9. The method of claim 1, wherein the at least one passageway through the bone graft is two passageways through the bone graft.

10. The method of claim 9, wherein the two passageways through the bone graft are spaced about 10 mm apart.

11. The method of claim 1, wherein the at least one passageway through the bone graft has a diameter of about 3 mm.

12. The method of claim 1, wherein forming at least one passageway through the glenoid comprises drilling at least one passageway through the glenoid with a drill.

13. The method of claim 12, wherein the drill is a 2.8 mm sleeved drill.

14. The method of claim 1, wherein the at least one passageway through the glenoid is two passageways through the glenoid.

15. The method of claim 14, wherein the two passageways through the glenoid are spaced about 10 mm apart.

16. The method of claim 1, wherein a distance between the at least one passageway through the bone graft and another passageway through the bone graft is substantially equal to a distance between the at least one passageway through the glenoid and another passageway through the glenoid.

17. The method of claim 1, wherein the at least one passageway through the glenoid is placed about 4 mm to about 5 mm on center below a cortical edge of the anterior surface of the glenoid.

18. The method of claim 12, wherein drilling at least one passageway through the glenoid with a drill comprises passing the drill through a billet secured to the posterior surface of the glenoid.

19. The method of claim 18, wherein securing the billet to the posterior surface of the glenoid comprises securing the billet with a glenoid guide.

20. The method of claim 1, wherein drawing the suture posteriorly comprises drawing a guide wire to which the suture has been secured posteriorly.

21. The method of claim 1, wherein securing the second end of the suture to the second implant comprises tying a knot in the suture.

22. The method of claim 21, wherein the knot is a Nice Knot.

23. The method of claim 1, wherein the second implant is a round endbutton.

24. A method of securing a bone graft to a glenoid, the method comprising:
forming first and second passageways through a bone graft having a cortical side and an opposing cancellous side;
forming first and second passageways through a glenoid of a patient, from a posterior surface to an anterior surface, a distance between the two passages through the glenoid being substantially the same as a distance between the two passages through the bone graft;
positioning a first implant secured to a first end of a first suture on the cortical side of the bone graft by drawing the first suture posteriorly through the first passageway in the bone graft and the first passageway in the glenoid;
positioning a second implant secured to a first end of a second suture on the cortical side of the bone graft by drawing the second suture posteriorly through the second passageway in the bone graft and the second passageway in the glenoid;
attaching a third implant to a second end of the first suture near the posterior surface of the glenoid;
attaching a fourth implant to a second end of the second suture near the posterior surface of the glenoid; and
tensioning the first and second sutures such that the third and fourth implants are urged into contact with the posterior surface of the glenoid and the bone graft is secured to the anterior surface of the glenoid.

25. The method of claim 24, wherein the first and second implants are the same.

26. The method of claim 24, wherein the third and fourth implants are the same.

27. The method of claim 24, wherein the bone block is one of an autograft, an allograft, or a synthetic.

28. The method of claim 24, wherein the two passageways through the glenoid are spaced about 10 mm apart.

29. The method of claim 24, further comprising positioning a hook end of an arm of a drill guide on the anterior surface of the glenoid, the drill guide comprising at least one bullet for passage of a drill.

30. The method of claim 24, wherein the two passageways in the bone graft are formed about 5 mm from an edge of the bone graft.
